(19) United States
(12) Patent Application Publication
(10) Pub. No.: US 2026/0002878 A1
(43) Pub. Date: Jan. 1, 2026

(54) HUMIDITY DYNAMICS SENSOR AND USE

(71) Applicants: Rutgers, The State University of New Jersey, New Brunswick, NJ (US); Texas A&M Innovation, College Station, TX (US)

(72) Inventors: Qingze ZOU, Bridgewater, NJ (US); Zezhou ZHANG, Piscataway, NJ (US); Jun ZOU, College Station, TX (US); Juan DONG, Piscataway, NJ (US); Shuangliang LI, College Station, TX (US); Aobo HUANG, Highland Park, NJ (US); Mary WHELAN, New Brunswick, NJ (US)

(21) Appl. No.: 19/245,710

(22) Filed: Jun. 23, 2025

Related U.S. Application Data
(60) Provisional application No. 63/664,804, filed on Jun. 27, 2024.

(51) Int. Cl. G01N 21/84; G01N 21/01; G01N 33/00; G01N 33/18

(52) U.S. Cl. CPC G01N 21/84; G01N 21/01; G01N 33/004; G01N 33/18; G01N 2021/0143; G01N 2021/8466

(57) ABSTRACT

Techniques are provided for sensing and using humidity dynamics. In a first set of embodiments, a humidity dynamics sensor includes a chamber that is open only at one wall opening. The sensor also includes a pair of humidity sensors disposed in a wall of the chamber at different distances from the wall opening. Each sensor is configured to measure humidity inside the chamber. The sensor also includes a gasket surrounding the wall opening of the chamber. At least a portion of the wall of the chamber is transparent to at least a portion of photosynthetically active optical wavelengths. The gasket is configured to form an airtight seal with a surface of a subject.

### CROSS-REFERENCE TO RELATED APPLICATIONS

This application claims benefit of Provisional Appln. 63/664,804, filed Jun. 27, 2024, the entire contents of which are hereby incorporated by reference as if fully set forth herein, under 35 U.S.C. § 119(e).

### STATEMENT OF GOVERNMENTAL INTEREST

This invention was made with government support under Grant Nos. 1851907 and 1952823 awarded by the National Science Foundation. The government has certain rights in the invention.

### BACKGROUND

Stomata are micrometer sized valves in plant leaves, the temporally varying opening and closing of which is responsible for gas exchange, e.g., the intake of carbon dioxide and water vapor, and the expulsion of oxygen and water vapor, during photosynthesis. Existing devices on the market are configured for measuring stomata conductance. It is a measure of the stomata activity at steady-state, i.e., it measures the amount of water vapor released over a relatively long period of time compared to stomata openings and closings. They are not built for, and thereby, do not have the capability to measure the dynamic activities of stomata, i.e., how fast the stomata open or close upon environmental condition changes.

### SUMMARY

Techniques are provided for sensing and using humidity dynamics. In a first set of embodiments, a humidity dynamics sensor includes a chamber that is open only at one wall opening. The sensor also includes a pair of humidity sensors disposed in a wall of the chamber at different distances from the wall opening. Each sensor is configured to measure humidity inside the chamber. The sensor also includes a gasket surrounding the wall opening of the chamber. At least a portion of the wall of the chamber is transparent to at least a portion of photosynthetically active optical wavelengths. The gasket is configured to form an airtight seal with a surface of a subject, such as a plant leaf.

In some of embodiments of the first set, the humidity dynamics sensor includes a carbon dioxide sensor configured to measure carbon dioxide inside the chamber.

In some of embodiments of the first set, the humidity dynamics sensor includes a camera configured to capture an image of a surface of a leaf at the wall opening of the chamber. In some of these embodiments, the camera is disposed inside the chamber. In other embodiments with the camera, the camera is disposed in an optical conduit configured to provide a view of the surface of the leaf at the wall opening of the chamber. In some embodiments, the conduit includes an amplification lens. In some embodiments with the camera, the sensor includes a light source configured to illuminate the surface of the subject at the wall opening of the chamber.

In other sets of embodiments, a system or computer-readable medium or computer apparatus or a method is configured to use the humidity dynamics sensor.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example of a computer-based system, used according to an embodiment;

FIG. 2 is a block diagram that illustrates an example of an humidity dynamics sensor system, according to an embodiment;

FIG. 3 is a flow chart that illustrates an example of a method for using the humidity dynamics sensor of FIG. 2, according to an embodiment;

FIG. 4 is a block diagram that illustrates a computer system 400 upon which an embodiment of the invention may be implemented; and

FIG. 5 illustrates a chip set upon which an embodiment of the invention may be implemented.

### DETAILED DESCRIPTION

A method and apparatus are described for measuring humidity dynamics such as in the vicinity of a plant leaf surface. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of milliscale change in the vicinity of a plant leaf which refers to a small portion of the leaf rather than the whole leaf, over minutes and hours rather than in a steady state. However, the invention is not limited to this context. In other embodiments other time and space scale in the vicinity of leaves and other biological organisms, including in vitro and in vivo measurements of animal cells and tissues, plants, fungi and microbes, and other surfaces such as naturally occurring and man-made films and membranes.

### 1. OVERVIEW

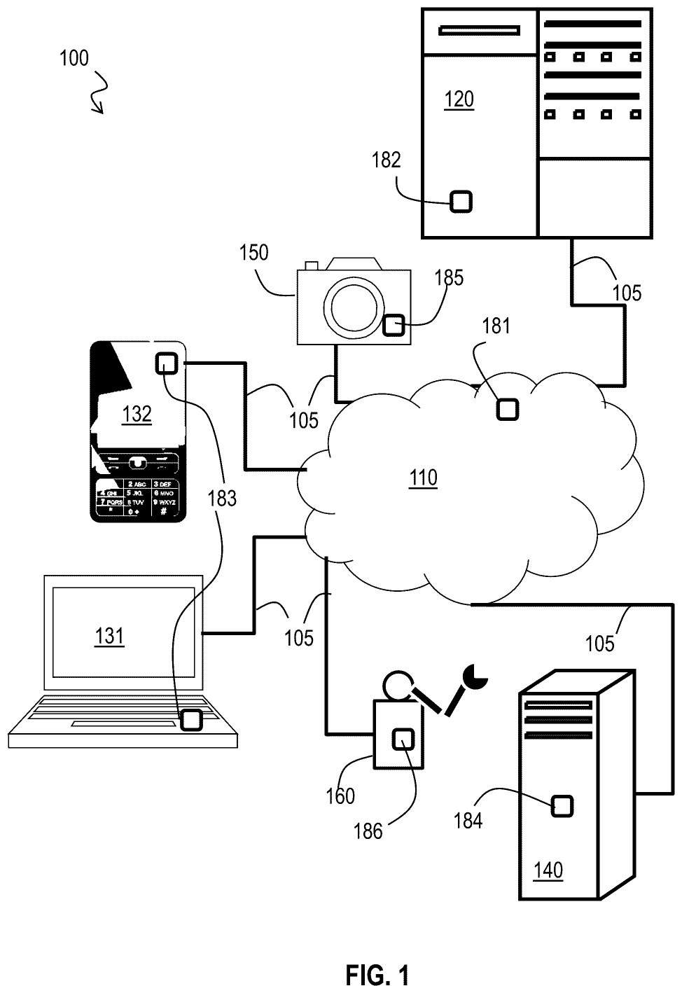

FIG. 1 is a block diagram that illustrates an example of a computer-based system, used according to an embodiment. The system includes a network 110 of interconnected communication devices using wired or wireless single channel or multichannel connections 105. User devices such as laptop 131 and mobile device 132 are connected to the network using one or more wired or wireless single channel or multichannel connections 105. One or more server nodes 120, often at different locations, also using one or more connections 105, provide services for devices connected to the system 100, and store data in one or more special storage nodes 140, often at different locations, also using one or more connections 105. In addition, in some systems, one or more sensors 150 (such as digital cameras, telescopes, laser and radar detectors, medical imagers, patient vital signs monitors, and the humidity dynamics sensors described herein, etc.) respond to physical phenomena with signals that are converted to data transmitted over connections 105 to other devices in the system 100. In addition, in some systems, one or more actuators 160 (such as assembly line robots, 2D and 3D printers, lasers, radiation sources, etc.) produce physical phenomena in response to signals received as data transmitted over connections 105 from other devices in the system 100. Each of these components include one or more software modules that operate the device and communicate with other devices in the systems, as represented by the module 181 for the network 110, module 182 for server(s) 120, module 183 for user device laptop 131 or mobile device 132, module 184 for storage node(s) 140, module 185 for sensor 150, and module 186 for actuator 160. The software modules 181, 182, 183, 184, 185 and 185 are collectively referenced herein as software modules 180.

Although processes, equipment, and data structures are depicted in FIG. 1 as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts.

### 1.1 Structure of Humidity Dynamics Sensor

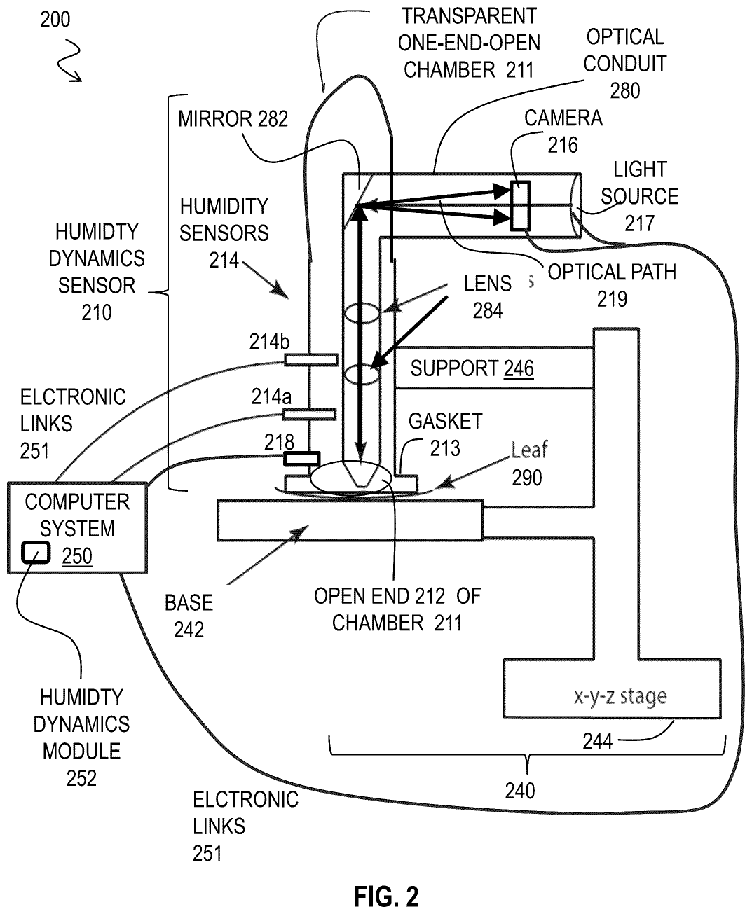

FIG. 2 is a block diagram that illustrates an example of a humidity dynamics sensor system 200, according to an embodiment. Although shown for purposes of illustration of use, a subject plant leaf 290 is not part of the system 200. The system 200 includes a humidity dynamics sensor 210, a support system 240 and a computer system 250.

The humidity dynamics sensor 210 includes a chamber 211 that is open only at one wall opening 212. At least a portion of a wall of chamber 211 is transparent to at least a portion of photosynthetically active optical wavelengths. The humidity dynamics sensor 210 also includes a pair of humidity sensors 214a and 214b (collectively sensor pair 214) disposed in the wall of the chamber 211 at different distances from the wall opening 212. Each sensor 214a and 214b is configured to measure humidity inside the chamber. In some embodiments each sensor 214a and 214b is a microelectromechanical system (MEMS) relative humidity senor. The humidity dynamic sensor 210 also includes a gasket 213 surrounding the wall opening 212 of chamber 211. Gasket 213 is configured to form an airtight seal with a surface of a subject plant leaf 290. The gasket 213 is transparent and may be made of a silicon polymer, such as polyimethylsiloxane (PDMS), for example. Thus, the inside of chamber 211 is separated from an external environment and not subject to air currents that can conflate the humidly profile above a subject plant leaf 290.

In some embodiments, the humidity dynamics sensor 210 includes a carbon dioxide sensor 218 configured to measure carbon dioxide inside chamber 211.

Some uses of the humidity dynamics sensor 210 include using a camera to capture an image of at least a portion of a surface of a subject plant leaf within the wall opening 212 of chamber 211. Because at least a portion of the chamber 211 wall is transparent, the image capture can be achieved by an external light source 217, external camera 215, and some optical conduit 280 passing light from the source 217 to the surface of the subject leaf 290 inside the wall opening 212 of the chamber 211 and back to the camera 216. An optical conduit 280 can include any optical transmission elements including free space, clear materials, optical fiber, beam splitter, mirror and lens, such as mirror 282 and lenses 284, alone or in some combination. In some embodiments the light source 217 or camera 215 or both, and any associated optical conduit 280, is included within chamber 211 or walls thereof.

Thus, in some embodiments, the humidity dynamics sensor includes a camera configured to capture an image of a surface of a leaf at the wall opening of the chamber. In some of these embodiments, the camera is disposed inside the chamber. In other embodiments with the camera, the camera is disposed in an optical conduit configured to provide a view of the surface of the leaf at the wall opening of the chamber. In some embodiments with the camera, the sensor includes a light source configured to illuminate the surface of the leaf at the wall opening of the chamber.

Some uses of the humidity dynamics sensor 210 is to investigate the role of a plurality of stomata on a portion of a leaf, a so called milliscale, over the course of one or more hours during which light initiates photosynthesis in the subject plant leaf, at least for a portion of that duration. For such uses, the wall opening 212 of chamber 211 has an area selected in a range from 1 square millimeter (mm, 1 mm=10⁻³ meters) to 10 square centimeters (cm, 1 cm=10⁻² meters). The height of the chamber 2112 is proportionally selected in a range from 2 mm to 10 cm.

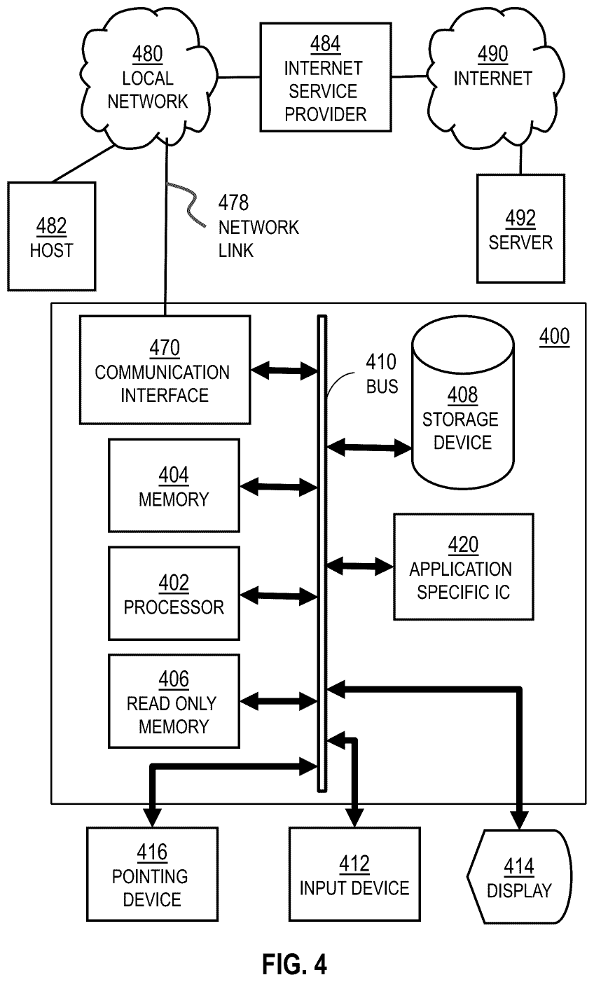

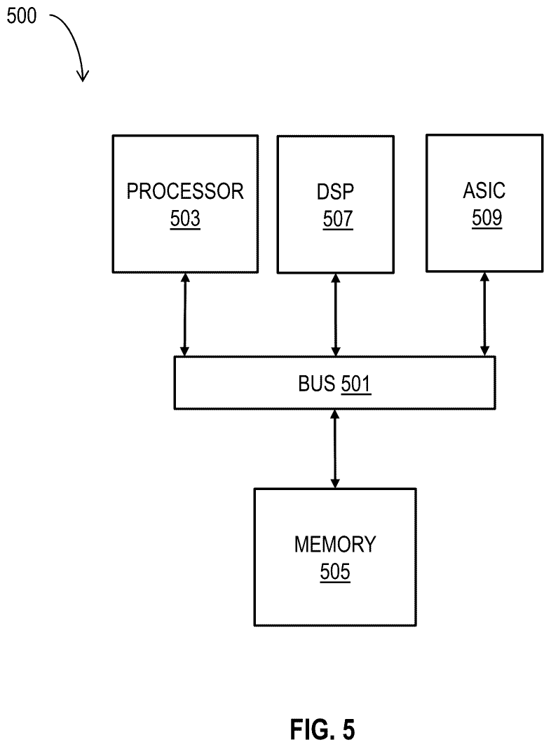

The computer system 250 includes one or more processors, such as depicted in FIG. 5, or computers in a network depicted in FIG. 1 and FIG. 4. The computer system 250 is in wired or wireless electronic communication with the humidity sensor pair 214 to control and record signals therefrom and is optionally in communication with the carbon dioxide sensor 218, light source 217 or camera 216, or some combination, to control and record signals therefrom. Distributed over one or more processors and storage devices in computer system 250 is a humidity dynamics module 252. The humidity dynamics module 252 is configured to determine the absolute humidity at the surface of a subject at the wall opening 212 of chamber 211 and, optionally, the physiographic features of the subject at a least a portion of the wall opening 212 of chamber 211 as captured in an image by camera 216. In some embodiments directed to a subject plant leaf, this includes determining any relationship between size or shape statistics, or both, of stomata in the image captured by camera 216 to the absolute humidity or carbon dioxide or light levels, or some combination, at the wall opening 212 of chamber 211.

The support system 240 includes a sensor support structure 246 configured to hold the humidity dynamics sensor 210 in place, a base 242 configured to hold a subject in position, and an x-y-z moveable stage to position base 242 relative to support 246, and thus enable the positioning of the wall opening 212 of chamber 211 in airtight contact with the subject, such as a subject plant leaf 290.

### 1.2 Method for Use of Humidity Dynamics Sensor

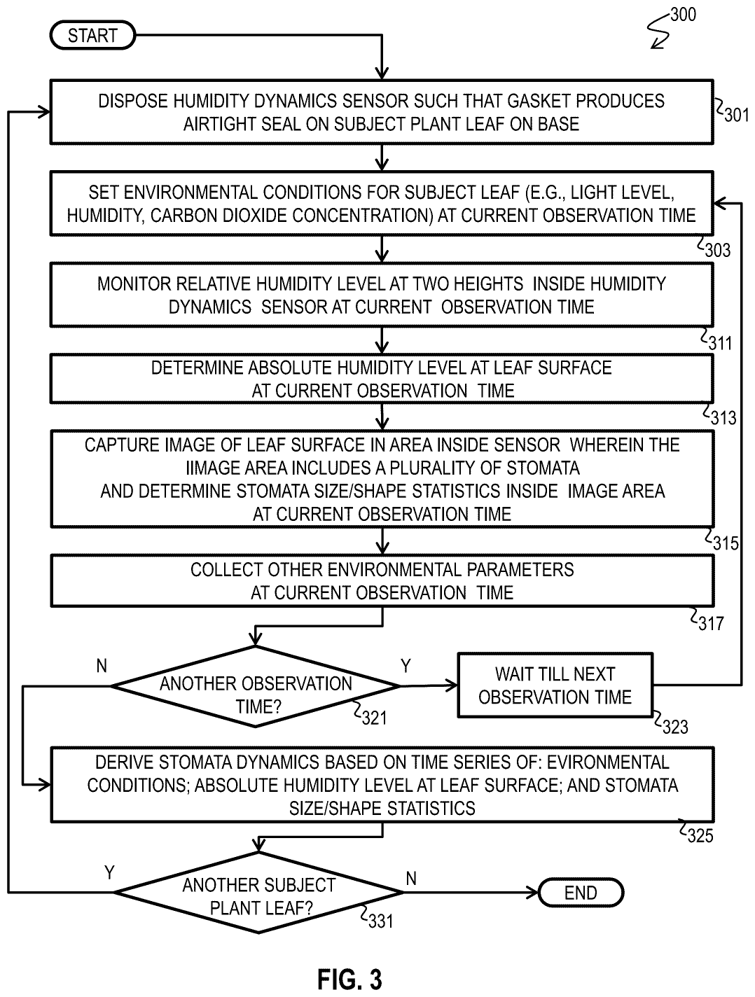

FIG. 3 is a flow chart 300 that illustrates an example of a method for using the humidity dynamics sensor of FIG. 2, according to an embodiment. Although steps are depicted in FIG. 3 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways. In this embodiment, the humidity dynamics system 200 is used to determine the dynamics of stomata in a plant leaf.

In step 301, the humidity dynamics sensor 210 is disposed such that gasket 213 produces airtight seal on subject plant leaf 290 on base 242, e.g., using moveable stage 244.

In step 303, environmental conditions are set (e.g., light level, humidity, carbon dioxide concentration) for a subject leaf at current observation time.

In step 311, relative humidity level is monitored at two heights inside humidity dynamics sensor at current observation time. In step 313, absolute humidity level is determined for leaf surface at current observation time, e.g., using a diffusion model as described in more detail below and relying on absence of external air currents, shear and turbulence inside chamber 211 of sensor 210.

In step 315, an image of leaf surface in area inside sensor is captured. The image area includes a plurality of stomata. Based on the captured image, stomata size/shape statistics inside image area is determined at current observation time.

In step 317, other environmental parameters, such as carbon dioxide, external light levels, external oxygen levels, external water vapor are collected at current observation time.

In step 321, it is determined whether there is another observation time. For example, in some embodiments, observations are made every second, or every ten seconds or every minute or every ten minutes or every hour over an observation period of several hours. If so, the control passes to step 323 to wait until the next observation time and then control passes back to step 303 to set the environmental conditions for the next observation time, and following steps, described above.

If it is determined in step 321 that there is not another observation time, then control passes to step 325. In step 325, stomata dynamics are derived based on time series of: environmental conditions; absolute humidity level at leaf surface; and stomata size/shape statistics, as described in more detail below. Control then passes to step 331.

In step 331, it is determined if there is another plant leaf to observe. If so, control passes back to step 301 and following described above. If not, the process ends.

### 2. EXAMPLE EMBODIMENTS

The humidity dynamics sensor system 200 and a corresponding methodology (using the system) advantageously measures the dynamic behavior of stomata at meso-scale during their opening and closing process. The humidity dynamics sensor system 200 utilizes a MEMS-based humidity sensor 210 to measure the water vapor released from the stomata during their opening process. The corresponding methodology is based on the diffusion model to quantify the amount of water vapor from the stomata from the measured humidity data as a function of time. The diffusion model may also be referred to as a fluidic-dynamics-based diffusion model or a water vapor diffusion model. The humidity dynamics sensor system 200 can be used for in-field stomatal measurement.

Two MEMS-based humidity sensors 214a, 214b are utilized to measure the humidity variations inside a transparent chamber or tube 211. This use of two MEMS sensors 214a, 214b is directly related to the methodology developed to quantify the water vapor release from the stomata at the leaf surface. This configuration brings additional benefits such as eliminating/avoiding the temperature fluctuation effect. Use of a transparent enclosure to create a stable measurement environment of small size that avoids/eliminates environmental disturbances such as air flow, and also allows the user to measure the response of stomata to environmental lighting condition (on/off). The measured relative humidity value is converted into absolute humidity as the measured data. This will also avoid the temperature effect on the measured result as relative humidity depends on the temperature. The diffusion model is configured to quantify the humidity variation at the leaf surface from the humidity measurement acquired by the two MEMS sensors 214a, 214b (located above the leaf 290 at given distances inside the transparent enclosure 211). By using this model, the time-varying water vapor release during the stomatal opening process can be accurately quantified.

The methodology of humidity measurement during stomatal opening based on the diffusion model will now be discussed. The methodology to quantify the humidity-based stomatal opening dynamics includes two parts: (1) the absolute humidity, and (2) the water-vapor release on the leaf surface (by using the quantified absolute humidity data). Absolute humidity (AH) (kg/m3) instead of relative humidity (RH) is measured as AH represents the absolute amount of water vapor released from the stomatal opening, while RH is influenced by the environmental temperature.

The following derivation is provided as an explanation for illustration purposes. Embodiments are not limited by the accuracy or comprehensiveness of the following derivation.

The RH is converted to AH via the following equation (1).

$$AH = \vert \frac{RH \times {P}_{s}}{{R}_{w} \times T \times 100} , \qquad ( 1 )$$

The variable Rw is the absolute temperature T [K] inside the transparent chamber 211 during the measurement, and Ps is the saturation water pressure.

To quantify the water vapor released from the stomatal opening process inside the tube chamber 211, the release process is modeled as a second-order diffusion process, and quantify the water vapor as the source from the two downstream water vapor measurements. The effect of temperature variation on the water vapor flow is negligible (as the variation inside the tube chamber 211 is small), as was the gravity effect. It is assumed that the water vapor inside the chamber 211 can be treated as ideal gas, and, thereby, obeys the Fickian diffusion process that can be described by Fick's law, as provided in equation (2).

$$\frac{\partial u}{\partial t} ( x , t ) = {α}^{2} \frac{\partial {u}^{2} ( x , t )}{\partial {x}^{2}} , for x ∈ [ 0 , {L}_{3} ] , \qquad ( 2 )$$

$$BC : u ( {L}_{1} , t ) = {h}_{1} ( t ) , u ( {L}_{2} , t ) = {h}_{2} ( t ) , {L}_{1} , {L}_{2} ∈ [ 0 , {L}_{3} ] ,$$

$$IC : u ( x , 0 ) = f ( x ) ,$$

In equation 2, u(x, t) denotes the AH at any given position x (w.r.t the leaf surface) and time instant t, L3 is the length of the tube chamber 211, and α [m²/s] is the water vapor diffusion coefficient in air, and u(L1, t) and u(L2, t) are the boundary conditions (BC) of the AH at any given position L1 and L2 (e.g., the two humidity sensors), respectively. The variable f(x) denotes the initial condition (IC) of the AH at a given position x when the light was switched on.

As the steady-state is reached before the light was switched on (i.e., at t=0), the initial humidity condition can be assumed the same everywhere inside the tube chamber 211, i.e., f(x)=H0 for x∈[0, L3]. Particularly, f(0)=h1(0)=h2(0) (the initial read-out of the two sensors). Thus, to account for the measurement variation between the two humidity sensors, H0 was estimated by the mean value between h1(0) and h2(0), as provided in equation (3):

$${H}_{0} = \frac{{h}_{1} \vert ( 0 ) + {h}_{2} ( 0 )}{2} . \qquad ( 3 )$$

By using the eigenfunction expansion method, the AH at any given location x∈[0, L3] (L3: length of the tube chamber) can be obtained from equation (4) below:

$$u ( x , t ) = w ( x , t ) + {\sum }_{n = 1}^{∞} [ \text{?} {\hat{S}}_{n} ( τ ) d τ + \text{?} {b}_{n} ] sin {λ}_{n} ( x - {L}_{1} ) , \qquad ( 4 )$$

$$where$$

$$w ( x , t ) = ( {h}_{1} ( t ) - {h}_{0} ( t ) ) ( \frac{x - {L}_{1}}{Δ L} ) + {h}_{1} ( t ) , with Δ L = {L}_{2} - {L}_{1} ,$$

$${\hat{S}}_{n} ( t ) = \frac{1}{Δ L} {\int }_{- Δ L}^{Δ L} - \frac{\partial w ( x , t )}{\partial t} sin [ {λ}_{n} ( x - {L}_{1} ) ] dx , {λ}_{n} = \frac{n π}{Δ L} ,$$

$$with n ∈ ℕ ( ℕ : the set of natural numbers ) ,$$

$$and {b}_{n} = \frac{2}{Δ L} {\int }_{0}^{Δ L} ( f ( x ) - w ( x , 0 ) ) sin [ {λ}_{n} ( x - {L}_{1} ) ] dx ,$$

$$\begin{matrix}u ( 0 , t ) = w ( 0 , t ) + {\sum }_{n = 1}^{∞} \{ \text{?} {\hat{S}}_{n} ( τ ) + \text{?} {b}_{n} d τ \} sin {λ}_{n} ( - {L}_{1} ) \\ \approx w ( 0 , t ) + \text{?} \{ \text{?} {\hat{S}}_{n} ( τ ) + \text{?} {b}_{n} d τ \} sin {λ}_{n} ( - {L}_{1} ) \\ w ( 0 , t ) + γ ( \text{?} )\end{matrix} , \qquad ( 5 )$$

$$\text{?} \text{indicates text missing or illegible when filed}$$

By using the AH u(x,t) computed in step [0043], the transpiration rate (Rtr(t)) from the stomata opening process inside the chamber can be further calculated as following

$${R}_{tr} ( t ) = \frac{d ( {J}_{w} ( t ) )}{dt} , with {J}_{w} ( t ) = {\int }_{0}^{{L}_{2}} u ( x , t ) dx . \qquad ( 6 )$$

Using the above computed transpiration rate as a function of time during the stomata opening process, the dynamic stomata conductance (Gs(t)) can be further calculated as

$${G}_{s} ( t ) = \frac{{R}_{ir} ( t ) {P}_{a}}{( 1 - RH ( t ) / 100 ) {P}_{w , a}} , \qquad ( 7 )$$

where Pw,a [kPa] is the saturation vapor pressure, Pa [kPa] is the air pressure.

Thus, in summary, the dynamic transpiration rate and stomata conductance can be obtained by first, converting the RH data measured by the two sensors via equation (3) to the corresponding AH, h1(t) and h2(t), then using the measured AH as the BC in equation (4) to quantify the water vapor release at the leaf ω(0, t), and finally, computing the AH at the leaf via equation (5), computing the transpiration rate via equation (6), and then the stomata conductance via Eq. (7).

Embodiments of this innovation can be used by researchers in a broad range of areas in plant biology and agriculture science and engineering. In plant biology, researchers/scientists working on stomata related research, including genetics, physiology and pathology aspects of stomata, and environmental scientists can study the plant-ecosystems related to stomata of plants. At commercial enterprises, companies' researchers/scientists can focus on crop breeding to optimize the growth and products of crops in stressful environment, e.g., breed type of crops to have swift stomatal (open and close) response upon sudden weather changes such as extreme heat waves and/or storms. Precision agriculture companies/scientists/researchers can use the real-time, in-field stomata dynamics response to accurately regulate water irrigation, lighting (for green house environment) and other growth control means.

The example humidity dynamics sensor system 200 measures dynamic response of stomata, i.e., how fast the stomata can open upon environmental change such as light, heat, CO₂ level and other factors. This provides an important complementary knowledge to what the existing devices/instruments can measure, and thereby, offers more complete knowledge to the measurement of stomata behaviors.

Including optical measurements allows the device to perform in-field measurements of stomata behavior in the plant's nature environment, e.g., it can be easily deported and set up to measure the stomata dynamics on plant's leaf in the natural environment (such as in crop fields or forest), with minor disturbance to the leaf function. The device will measure simultaneously both the morphological changes and the water vapor release of stomata upon environmental condition changes (including lighting condition, temperature and CO₂ level).

By incorporating external sensors in some embodiments, the humidity dynamics sensor system 200 will also monitor and record the environmental conditions during the measurement, including the air temperature, the CO₂ density, and the environment lighting conditions. 1. The design may further include algorithms to minimize adverse effects of environmental disturbances such as wind, and will use the real-time measured environmental data (including temperature, CO₂ density and light density) into the characterization of stomatal conductance (water vapor release).

Some embodiments include algorithms to accurately quantify the instantaneous dynamics of stomata. Some embodiments also include image processing and pattern recognition algorithms to quantify the opening status of stomata, e.g., the real-time aperture size of each stomata and the number of stomata in the stomata image, and use it in the quantification of water vapor release. This will provide a much more accurate measurement of dynamic stomata conductance.

More particularly, embedded within the computing system 250 is a suite of algorithms, as described below. One of the algorithms is for absolute water vapor release quantification. The measured water vapor signal will be converted to the absolute humidity (i.e., the amount of water vapor release) changes during the measurement period, based on the diffusion model of the water vapor flow inside the measurement chamber 211.

A set of algorithms is for stomata identification and geometric shape quantification. These algorithms automatically identify the stomata from the captured images, and then automatically quantifies the size of the each stomata in each captured image. Together, this will allow the total stomata opening area to be quantified at each sampled time instant, which, in turn, will be used to quantify the normalized real-time water vapor release for each measured plant, i.e., the measured absolute water vapor release divided by the total stomata opening area. This information, which is currently not available in any existing stomata conductance measurement instruments, provides a much more accurate characterization of the stomata behavior.

Feature extraction algorithms, such as the circle hough transform (for circular and ellipse-like shapes) and the scale-invariant feature transform may be used to identify the stomata and quantify their sizes. Machine-learning based pattern recognition algorithms may also be included in the suite of algorithms.

Yet another set of algorithms is for signal filtering to eliminate other environmental disturbance and enhance signal to noise ratio. These algorithms filter the measured signals to reduce and avoid other adverse environmental effects, and enhance the signal-to-noise ratio. These algorithms may be based on data-driven extended Kalman filtering techniques and Wiener filtering techniques.

Stomatal conductance (gs) measured the rate of CO₂ entering or water vapor exiting through a plant's stomata. It serves as an indication of stomatal density, aperture, and size. While commercially available portable photosynthesis systems provide valuable insights into the collective outcomes of stomatal movement within a specific leaf area, there exists a gap in the market for an instrument capable of real-time monitoring of dynamic changes in individual stomata. Such a groundbreaking instrument would offer increased repeatability and confidence in research findings. The absence of a tool that allows real-time observation of individual stomatal behavior hampers one's ability to fully comprehend the intricacies of stomatal function. By enabling the direct monitoring of stomatal open/close status and assessing individual differences within a population, this innovative instrument significantly advances our understanding of stomatal conductance. It elucidates the direct linkages and influences between individual stomatal behaviors and the resulting collective stomatal conductance, paving the way for more precise and comprehensive plant physiological studies. Measuring both the water vapor release and the shape/size changes of the stomata simultaneously provides the missing information/knowledge to correlate these two most important aspects of stomata behavior together. Making this link will provide a key improvement for many agricultural technologies.

### 3. COMPUTATIONAL HARDWARE OVERVIEW

FIG. 4 is a block diagram that illustrates a computer system 400 upon which an embodiment of the invention may be implemented. Computer system 400 includes a communication mechanism such as a bus 410 for passing information between other internal and external components of the computer system 400. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 400, or a portion thereof, constitutes a means for performing one or more steps of one or more methods described herein.

A sequence of binary digits constitutes digital data that is used to represent a number or code for a character. A bus 410 includes many parallel conductors of information so that information is transferred quickly among devices coupled to the bus 410. One or more processors 402 for processing information are coupled with the bus 410. A processor 402 performs a set of operations on information. The set of operations include bringing information in from the bus 410 and placing information on the bus 410. The set of operations also typically include comparing two or more units of information, shifting positions of units of information, and combining two or more units of information, such as by addition or multiplication. A sequence of operations to be executed by the processor 402 constitutes computer instructions.

Computer system 400 also includes a memory 404 coupled to bus 410. The memory 404, such as a random access memory (RAM) or other dynamic storage device, stores information including computer instructions. Dynamic memory allows information stored therein to be changed by the computer system 400. RAM allows a unit of information stored at a location called a memory address to be stored and retrieved independently of information at neighboring addresses. The memory 404 is also used by the processor 402 to store temporary values during execution of computer instructions. The computer system 400 also includes a read only memory (ROM) 406 or other static storage device coupled to the bus 410 for storing static information, including instructions, that is not changed by the computer system 400. Also coupled to bus 410 is a non-volatile (persistent) storage device 408, such as a magnetic disk or optical disk, for storing information, including instructions, that persists even when the computer system 400 is turned off or otherwise loses power.

Information, including instructions, is provided to the bus 410 for use by the processor from an external input device 412, such as a keyboard containing alphanumeric keys operated by a human user, or a sensor. A sensor detects conditions in its vicinity and transforms those detections into signals compatible with the signals used to represent information in computer system 400. Other external devices coupled to bus 410, used primarily for interacting with humans, include a display device 414, such as a cathode ray tube (CRT) or a liquid crystal display (LCD), for presenting images, and a pointing device 416, such as a mouse or a trackball or cursor direction keys, for controlling a position of a small cursor image presented on the display 414 and issuing commands associated with graphical elements presented on the display 414.

In the illustrated embodiment, special purpose hardware, such as an application specific integrated circuit (IC) 420, is coupled to bus 410. The special purpose hardware is configured to perform operations not performed by processor 402 quickly enough for special purposes. Examples of application specific ICs include graphics accelerator cards for generating images for display 414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 400 also includes one or more instances of a communications interface 470 coupled to bus 410. Communication interface 470 provides a two-way communication coupling to a variety of external devices that operate with their own processors, such as printers, scanners and external disks. In general the coupling is with a network link 478 that is connected to a local network 480 to which a variety of external devices with their own processors are connected. For example, communication interface 470 may be a parallel port or a serial port or a universal serial bus (USB) port on a personal computer. In some embodiments, communications interface 470 is an integrated services digital network (ISDN) card or a digital subscriber line (DSL) card or a telephone modem that provides an information communication connection to a corresponding type of telephone line. In some embodiments, a communication interface 470 is a cable modem that converts signals on bus 410 into signals for a communication connection over a coaxial cable or into optical signals for a communication connection over a fiber optic cable. As another example, communications interface 470 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 470 sends and receives electrical, acoustic or electromagnetic signals, including infrared and optical signals, that carry information streams, such as digital data.

The term computer-readable medium is used herein to refer to any medium that participates in providing information to processor 402, including instructions for execution. Such a medium may take many forms, including, but not limited to, non-volatile media, volatile media and transmission media. Non-volatile media include, for example, optical or magnetic disks, such as storage device 408. Volatile media include, for example, dynamic memory 404. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 402, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 402, except for carrier waves and other signals.

Logic encoded in one or more tangible media includes one or both of processor instructions on a computer-readable storage media and special purpose hardware, such as ASIC 420.

Network link 478 typically provides information communication through one or more networks to other devices that use or process the information. For example, network link 478 may provide a connection through local network 480 to a host computer 482 or to equipment 484 operated by an Internet Service Provider (ISP). ISP equipment 484 in turn provides data communication services through the public, world-wide packet-switching communication network of networks now commonly referred to as the Internet 490. A computer called a server 492 connected to the Internet provides a service in response to information received over the Internet. For example, server 492 provides information representing video data for presentation at display 414.

The invention is related to the use of computer system 400 for implementing the techniques described herein. According to one embodiment of the invention, those techniques are performed by computer system 400 in response to processor 402 executing one or more sequences of one or more instructions contained in memory 404. Such instructions, also called software and program code, may be read into memory 404 from another computer-readable medium such as storage device 408. Execution of the sequences of instructions contained in memory 404 causes processor 402 to perform the method steps described herein. In alternative embodiments, hardware, such as application specific integrated circuit 420, may be used in place of or in combination with software to implement the invention. Thus, embodiments of the invention are not limited to any specific combination of hardware and software.

The signals transmitted over network link 478 and other networks through communications interface 470, carry information to and from computer system 400. Computer system 400 can send and receive information, including program code, through the networks 480, 490 among others, through network link 478 and communications interface 470. In an example using the Internet 490, a server 492 transmits program code for a particular application, requested by a message sent from computer 400, through Internet 490, ISP equipment 484, local network 480 and communications interface 470. The received code may be executed by processor 402 as it is received or may be stored in storage device 408 or other non-volatile storage for later execution, or both. In this manner, computer system 400 may obtain application program code in the form of a signal on a carrier wave.

Various forms of computer readable media may be involved in carrying one or more sequence of instructions or data or both to processor 402 for execution. For example, instructions and data may initially be carried on a magnetic disk of a remote computer such as host 482. The remote computer loads the instructions and data into its dynamic memory and sends the instructions and data over a telephone line using a modem. A modem local to the computer system 400 receives the instructions and data on a telephone line and uses an infra-red transmitter to convert the instructions and data to a signal on an infra-red a carrier wave serving as the network link 478. An infrared detector serving as communications interface 470 receives the instructions and data carried in the infrared signal and places information representing the instructions and data onto bus 410. Bus 410 carries the information to memory 404 from which processor 402 retrieves and executes the instructions using some of the data sent with the instructions. The instructions and data received in memory 404 may optionally be stored on storage device 408, either before or after execution by the processor 402.

FIG. 5 illustrates a chip set 500 upon which an embodiment of the invention may be implemented. Chip set 500 is programmed to perform one or more steps of a method described herein and includes, for instance, the processor and memory components described with respect to FIG. 4 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 500, or a portion thereof, constitutes a means for performing one or more steps of a method described herein.

In one embodiment, the chip set 500 includes a communication mechanism such as a bus 501 for passing information among the components of the chip set 500. A processor 503 has connectivity to the bus 501 to execute instructions and process information stored in, for example, a memory 505. The processor 503 may include one or more processing cores with each core configured to perform independently. A multi-core processor enables multiprocessing within a single physical package. Examples of a multi-core processor include two, four, eight, or greater numbers of processing cores. Alternatively, or in addition, the processor 503 may include one or more microprocessors configured in tandem via the bus 501 to enable independent execution of instructions, pipelining, and multithreading. The processor 503 may also be accompanied with one or more specialized components to perform certain processing functions and tasks such as one or more digital signal processors (DSP) 507, or one or more application-specific integrated circuits (ASIC) 509. A DSP 507 typically is configured to process real-world signals (e.g., sound) in real time independently of the processor 503. Similarly, an ASIC 509 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 503 and accompanying components have connectivity to the memory 505 via the bus 501. The memory 505 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 505 also stores the data associated with or generated by the execution of one or more steps of the methods described herein.

### 4. ALTERNATIVES, DEVIATIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 5. REFERENCES

Each of the references cited below is hereby incorporated by reference as if fully quoted herein, except for terminology inconsistent with that used herein.
[1] Andree Tuzet. Stomatal conductance, photosynthesis and transpiration, modelling, 2011.
[2] Josef Urban, Miles Ingwers, Mary Anne McGuire, and Robert O Teskey. Stomatal conductance increases with rising temperature. Plant signaling & behavior, 12(8):e1356534, 2017.
[3] Keith A Mott. Do stomata respond to co2 concentrations other than intercellular? Plant physiology, 86(1):200-203, 1988.
[4] Ken-ichiro Shimazaki, Michio Doi, Sarah M Assmann, and Toshinori Kinoshita. Light regulation of stomatal movement. Annu. Rev. Plant Biol., 58:219-247, 2007.
[5] David Peak and Keith A Mott. A new, vapour-phase mechanism for stomatal responses to humidity and temperature. Plant, cell & environment, 34(1):162-178, 2011.
[6] Tracy Lawson and Michael R Blatt. Stomatal size, speed, and responsiveness impact on photosynthesis and water use efficiency. Plant physiology, 164(4):1556-1570, 2014.
[7] Alistair M Hetherington and F Ian Woodward. The role of stomata in sensing and driving environmental change. Nature, 424(6951):901-908, 2003.
[8] Yoshiyuki Murata, Izumi C Mori, and Shintaro Munemasa. Diverse stomatal signaling and the signal integration mechanism. Annual Review of Plant Biology, 66:369-392, 2015.
[9] Brian Thomas. Encyclopedia of applied plant sciences. Academic Press, 2016.
[10] https://www.metergroup.com/en/meter-environment/products/sc-1-leafporometer-stomatal-conductance. Accessed: 2023 May 1.
[11] https://delta-t.co.uk/product/ap4/overview. Accessed: 2023 May 1.
[12] https://www.licor.com/env/products/photosynthesis/LI-6800/. Accessed: 2023 May 1.
[13] Jack S A Matthews, Silvere R M Vialet-Chabrand, and Tracy Lawson. Diurnal variation in gas exchange: the balance between carbon fixation and water loss. Plant Physiology, 174(2):614-623, 2017.
[14] Silvere R M Vialet-Chabrand, Jack S A Matthews, Lorna McAusland, Michael R Blatt, Howard Griffiths, and Tracy Lawson. Temporal dynamics of stomatal behavior: modeling and implications for photosynthesis and water use. Plant physiology, 174(2):603-613, 2017.
[15] Tracy Lawson and Silv\grave{ }ere Vialet-Chabrand. Speedy stomata, photosynthesis and plant water use efficiency. New Phytologist, 221(1):93-98, 2019.
[16] James W Clark, Brogan J Harris, Alexander J Hetherington, Natalia Hurtado-Castano, Robert A Brench, Stuart Casson, Tom A Williams, Julie E Gray, and Alistair M Hetherington. The origin and evolution of stomata. Current Biology, 32(11):R539-R553, 2022.
[17] Jack S A Matthews, Silvere Vialet-Chabrand, and Tracy Lawson. Role of blue and red light in stomatal dynamic behaviour. Journal of Experimental Botany, 71(7):2253-2269, 2020.
[18] Paul E Waggoner and Israel Zelitch. Transpiration and the stomata of leaves: Water loss through leaf pores is controlled by pore size, which varies with environment and chemical sprays. Science, 150(3702):1413-1420, 1965.

1. A humidity dynamics sensor comprising:
a chamber that is open only at one wall opening;
a pair of humidity sensors disposed in a wall of the chamber at different distances from the wall opening, each sensor configured to measure humidity inside the chamber; and
a gasket surrounding the wall opening of the chamber,
wherein
at least a portion of the wall of the chamber is transparent to at least a portion of photosynthetically active optical wavelengths, and
the gasket is configured to form an airtight seal with a surface of a subject.

2. The humidity dynamics sensor as recited in claim 1, further comprising a carbon dioxide sensor configured to measure carbon dioxide inside the chamber.

3. The humidity dynamics sensor as recited in claim 1, further comprising a camera configured to capture an image of a surface of the subject at the wall opening of the chamber.

4. The humidity dynamics sensor as recited in claim 3, wherein the camera is disposed inside the chamber.

5. The humidity dynamics sensor as recited in claim 3, wherein the camera is disposed in an optical conduit configured to provide a view of the surface of the subject at the wall opening of the chamber.

6. The humidity dynamics sensor as recited in claim 3 further comprising a light source configured to illuminate the surface of the subject at the wall opening of the chamber.

7. The humidity dynamics sensor as recited in claim 1, wherein the gasket comprises a transparent silicone polymer.

8. A system comprising:
the humidity dynamics sensor of claim 1; and
a computer system configured to record time series measurements from the pair of humidity sensors and determine a time series of values for absolute humidity at the wall opening of the chamber.

9. The system as recited in claim 8, wherein the subject comprises a leaf, and the absolute humidity represents an absolute amount of water vapor released from stomatal openings within the leaf.

10. A system comprising:
the humidity dynamics sensor of claim 3; and
a computer system configured to
record a first time series of measurements from the pair of humidity sensors, and
determine a second time series of statistics of stomata size or shape or both captured by the camera.

11. The system as recited in claim 10, wherein the computer system is further configured to determine dynamics of stomata in a surface of a leaf at the wall opening of the chamber.

12. A non-transitory computer-readable medium carrying one or more sequences of instructions, wherein execution of the one or more sequences of instructions by one or more processors causes the one or more processors to perform the steps of:
recording time series measurements from the pair of humidity sensors of the humidity dynamics sensor of claim 1 and
determining a time series of values for absolute humidity at the wall opening of the chamber.

13. A non-transitory computer-readable medium carrying one or more sequences of instructions, wherein execution of the one or more sequences of instructions by one or more processors causes the one or more processors to perform the step of:
recording a first time series of measurements from the humidity dynamics sensor of claim 3, and
determining a second time series of statistics of stomata size or shape or both captured by the camera of the humidity dynamics sensor.

14. An apparatus comprising:
at least one processor; and
at least one memory including one or more sequences of instructions,
the at least one memory and the one or more sequences of instructions configured to, with the at least one processor, cause the apparatus to perform at least the following,
recording time series measurements from the pair of humidity sensors of the humidity dynamics sensor of claim 1, and
determining a time series of values for absolute humidity at the wall opening of the chamber.

15. An apparatus comprising:
at least one processor; and
at least one memory including one or more sequences of instructions,
the at least one memory and the one or more sequences of instructions configured to, with the at least one processor, cause the apparatus to perform at least the following,
recording time series measurements from the pair of humidity sensors of the humidity dynamics sensor of claim 3, and
determining a second time series of statistics of stomata size or shape or both captured by the camera of the humidity dynamics sensor.

16. A method comprising:
positioning a subject on a base;
positioning the humidity dynamics sensor of claim 3 on the subject to produce an airtight seal with a surface of the subject;
setting environmental conditions for the subject, and
determining a time series of values for absolute humidity at the surface of the subject.

17. The method as recited in claim 16, wherein the subject comprises a leaf, and the absolute humidity represents an absolute amount of water vapor released from stomatal openings within the leaf.

18. A method comprising:
positioning a subject on a base;
positioning the humidity dynamics sensor of claim 3 on the subject to produce an airtight seal with a surface of the subject;
setting environmental conditions for the subject;
recording a first time series of measurements from the pair of humidity sensors of the humidity dynamics sensor, and
determining a second time series of statistics of stomata size or shape or both captured by the camera of the humidity dynamics sensor.

19. The method as recited in claim 18, wherein the subject comprises a leaf, and comprising determining dynamics of stomata in a surface of the leaf.
